SEQUENCE LISTING (8 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 8 aa, Artificial Sequence
  Description of Artificial Sequence Synthetic peptide
DAEFRHDS

SEQ ID NO: 3 - protein, 11 aa, Artificial Sequence
  Description of Artificial Sequence Synthetic peptide
DAEFRHDSGYE

SEQ ID NO: 4 - protein, 16 aa, Artificial Sequence
  Description of Artificial Sequence Synthetic peptide
DAEFRHDSGYEVHHQK

SEQ ID NO: 5 - protein, 9 aa, Artificial Sequence
  Description of Artificial Sequence Synthetic peptide
EFRHDSGYE

SEQ ID NO: 6 - protein, 9 aa, Artificial Sequence
  Description of Artificial Sequence Synthetic peptide
EFRHDSGYE

SEQ ID NO: 7 - protein, 6 aa, Artificial Sequence
  Description of Artificial Sequence Synthetic peptide
EVHHQK

SEQ ID NO: 8 - protein, 6 aa, Artificial Sequence
  Description of Artificial Sequence Synthetic peptide
EVHHQK

(19) United States
(12) Patent Application Publication
(10) Pub. No.: US 2015/0024512 A1
(43) Pub. Date: Jan. 22, 2015

(54) METHOD FOR SELECTIVELY QUANTIFYING A-BETA AGGREGATES

(71) Applicant: FORSCHUNGSZENTRUM JUELICH GmbH, Juelich (DE)

(72) Inventors: Dieter Willbold, Juelich (DE); Susanne Aileen Funke, Sonnefeld (DE); Lei Wang-Dietrich, Wiesbaden (DE); Eva Birkmann, Korschenbroich (DE); Oliver Bannach, Duesseldorf (DE)

(21) Appl. No.: 14/366,952

(22) PCT Filed: Dec. 21, 2012
(86) PCT No.: PCT/EP2012/076552

§ 371 Date: Jun. 19, 2014

(30) Foreign Application Priority Data
Dec. 23, 2011 (DE) 10 2011 057 021.7

(51) Int. Cl. G01N 33/552; G01N 33/543

(52) U.S. Cl. CPC G01N 33/552; G01N 33/54393; G01N 2500/02; G01N 2800/2821; G01N 2458/00; G01N 2333/4709
USPC 436/501; 530/389.1

(57) ABSTRACT

The invention relates to methods for selectively quantifying A-beta aggregates, comprising the immobilization of anti-A-beta antibodies on a substrate, application of the sample to be tested onto the substrate, addition of probes labeled for detection, which mark these by specific binding to A-beta aggregates and detection of the marked aggregates.

The invention relates to methods for selectively quantifying A-beta aggregates comprising the immobilization of A-beta capture molecules on a substrate, application of the sample to be tested onto the substrate, addition of probes labeled for detection which by specific binding to A-beta aggregates mark these, and detection of the marked aggregates.

A-beta aggregates occur in Alzheimer's disease (AD, Alzheimer's dementia, Latin=Morbus Alzheimer). Together with Parkinson's disease for example, these belong to a heterogeneous group of clinical conditions the common criterion whereof is in many cases (but not exclusively) extracellular, systemic or local deposits of a protein specific in each case, mostly in the ordered conformation of beta sheet structure. In modern society, age-related dementia is an ever greater problem since owing to the increased life expectation ever more people are affected by it and the disease thus has repercussions on the social insurance systems and their financial viability.

Pathological aggregates from endogenous proteins, such as for example oligomers or fibrils, occur in many neurodegenerative diseases. In Alzheimer's dementia, for example, amyloid-beta peptide deposits (A-beta peptide deposits) are found in the brain and in Parkinson's disease synuclein deposits. The amyloid-beta peptide deposits (or peptide fibrils) are however merely the final stage of a process which begins with the cleavage of monomeric amyloid-beta peptides from APP (amyloid precursor protein), then forms neurotoxic amyloid-beta peptide oligomers and finally or alternatively ends with amyloid-beta peptide fibrils, deposited in plaques. Main pathological features of AD are the formation of senile or amyloid plaques, consisting of the A-beta peptide, and additional neurofibrillar deposits of the tau protein. The precursor protein of the A-beta peptide, APP, is located in the cell wall of neurones. Through proteolytic degradation and subsequent modification, A-beta fragments of various length and nature, such as for example A-beta 1-40, A-beta 1-42 or pGluA-beta 3-42 are formed from this. Monomeric A-beta peptides are also formed in the healthy body throughout life.

According to the amyloid cascade hypothesis from the 1990's, the A-beta deposits in the form of plaques are the triggers of the disease symptoms. In recent years, however, various studies are indicating that in particular the small, freely diffusing A-beta oligomers possess the greatest toxicity among all A-beta species and are responsible for the onset and progression of AD. Thus aggregates of the A-beta peptides are directly linked with AD pathogenesis.

At present, a reliable diagnosis of AD is only possible after the appearance of prominent clinical symptoms, and a reliability of at most 90% is assumed in this. The only previously certain diagnostic possibility at present exists only after the patient's death, through histological evidence of various changes in the brain.

Accordingly, there is a need for methods for the identification and quantitative estimation of A-beta aggregates, in particular of small, freely diffusing A-beta oligomers or aggregates.

Only a few methods for the characterization and quantification of pathogenic aggregates or oligomers in tissues and body fluids have so far been described.

Compounds which bind to Abeta and inhibit the aggregation thereof are for example known from Chafekar et al. (ChemBioChem 2007, 8, 1857-1864). These substances consist of parts of the Abeta peptide (KLVFF sequence) and are used for therapeutic purposes, and characterization and quantification of pathogenic aggregates or oligomers in tissues and body fluids is not performed with these.

At present, there are still no generally recognized criteria and/or identifications, so-called biomarkers, for AD. One approach for such biomarkers previously was the use of PET radioactive tracers for imaging methods, which is based on the assumption that the radioactively marked substances bind amyloid plaques and could thus after detection be a measure of the plaque deposition. In spite of an obvious connection between PET signal and disease, it was not previously possible to show that a reliable diagnosis is possible thereby, since many persons with no dementia also exhibit high tracer retention. Also disadvantageous for this method are the high costs and the necessary technical expenditure on instruments which are not available everywhere.

As a further approach, at present the quantities of various substances in the blood or spinal fluid (CSF) of patients are being studied and their usefulness as biomarkers analyzed. One of these substances is the A-beta peptide. So far, the determination of the content of monomeric Abeta in the spinal fluid of patients, possibly combined with the determination of the tau concentration seems the most reliable. However, there is such high variation of the values that no reliable diagnosis can be made for an individual by means of such biomarkers. The use of such a method is known from DE 89533623 T2. In spite of these different approaches, it has not so far been possible for any reliable biomarker to become established.

A further difficulty is that for the specific quantification of A-beta aggregates as opposed to A-beta monomers and/or the A-beta total content, only a few detection systems are so far available. As a possible detection system, ELISAs in which the A-beta oligomers are detected by means of antibodies are at present used. The antibodies used therein recognize either only quite specific types of A-beta oligomers or nonspecifically other oligomers which do not consist of A-beta peptides, but of quite different proteins, which has an adverse effect on the evaluation.

The use of ELISA-supported methods by means of conformer-specific antibodies is for example known from WO2005/018424 A2.

As a further detection method, sandwich ELISA measurements are used. Here A-beta-specific antibodies are used in order to immobilize A-beta molecules. The same antibodies are then also used for the detection. By this method, monomers result in no signal, since the antibody binding site is already occupied by the capture molecules. Specific signals are thus only created by dimers or larger oligomers. In the assessment however, such a method only enables the quantification of the sum of all aggregates present in a sample and not the characterization of individual aggregates. In order to reliably detect and quantify individual A-beta aggregates, the ELISA-supported method also lacks the sensitivity necessary for this. The use of such a sandwich ELISA method is known from WO2008/070229 A2.

The purpose of the present invention was to provide a biomarker for protein aggregation diseases, in particular AD, and an ultrasensitive method for quantifying and characterizing A-beta aggregates. Through characterization of the biomarker, i.e. determination of the number, quantity and/or size of this substance (biomarker) in an endogenous fluid or tissue, precise diagnosis of the disease and/or information about the course of the disease and the condition of the patient should be made possible. A further purpose of the present invention was to provide a method for selectively quantifying pathogenic aggregates which cause and/or characterize a protein aggregation disease, in particular of A-beta aggregates of any size and composition, A-beta oligomers and at the same time also small, freely diffusing A-beta oligomers.

This purpose is fulfilled by a method for selective quantification and/or characterization of A-beta aggregates comprising the following steps:
a) application of the sample to be tested onto the substrate,
b) addition of probes labeled for detection, which by specific binding to A-beta aggregates mark these and
c) detection of the marked aggregates, wherein step b) can be performed before step a).

Characterization of the A-beta aggregates or A-beta oligomers means determination of the form, size and/or composition.

In the sense of the present invention, the term A-beta monomer describes a peptide molecule which is a part of the amyloid precursor protein APP which is known under the name A-beta. Depending on the source species (man and/or animal) and processing, the precise amino acid sequence of an A-beta monomer can vary in length and nature.

In the sense of the present invention, the term A-beta oligomers describes both A-beta aggregates and also A-beta oligomers and also small, freely diffusing A-beta oligomers. Oligomer in the sense of the invention is a polymer formed from 2, 3, 4, 5, 6, 7, 8, 9, 10, 11, 12, 13, 14, 15, 16, 17, 18, 19 or 20 monomers or multiples thereof. Here, all A-beta monomers in an A-beta oligomer can be, but do not have to be, identical to one another.

Thus A-beta aggregates should be understood to mean both A-beta oligomers and also small, freely diffusing A-beta oligomers. This also includes aggregates, as for example fragments of fibrils, “protofibrils”, “ADDLSs” and p56* are described. It is essential for the present invention that with regard to size the A-beta aggregates are aggregates or polymers which can move in the body and are not because of their size immobilized in the body in the form of amyloid-beta peptide plaque deposits.

As the substrate, according to the invention a material is selected which possess as low as possible, nonspecific binding capacity, in particular with regard to A-beta oligomers.

In one implementation of the present invention, a glass substrate is selected.

The substrate can be coated with hydrophilic materials, preferably poly-D-lysine, polyethylene glycol (PEG) or dextran.

In one implementation of the present invention, the glass surface is hydroxylated and then activated with amino groups.

For the preparation of the substrate for coating, one or more of the following steps are performed:
washing of a glass substrate or a glass support in the ultrasonic bath or plasma cleaner, alternatively to this, incubate for at least 3 hours in 5M NaOH,
rinsing with water and subsequent drying under nitrogen,
immersion in a solution of concentrated sulfuric acid and hydrogen peroxide in the ratio 3:1 for the activation of the hydroxyl groups,
rinsing with water to neutral pH, then with ethanol and drying under a nitrogen atmosphere,
immersion in a solution with 3-aminopropyltriethoxysilane (APTES) (1-7%) in dry toluene or a solution of ethanolamine,
rinsing with acetone or DMSO and water and drying under a nitrogen atmosphere.

For the coating with dextran, preferably carboxymethyl dextran (CMD), the substrate is incubated with an aqueous solution of CMD (at a concentration of 10 mg/ml or 20 mg/ml) and optionally N-ethyl-N-(3-dimethylaminpropyl) carbodiimide (EDC), (200 mM) and N-hydroxysuccinimide (NHS), (50 mM) and then washed.

In one modification, the carboxymethyl dextran covalently bound to the glass surface, which has first been hydroxylated and then activated with amine groups, as described above.

As the substrate, microtiter plates preferably with glass bases, can also be used. Since with the use of polystyrene frames the use of concentrated sulfuric acid is not possible, the activation of the glass surface is effected in one practical modification of the invention analogously to Janissen et al. (Colloids Surf B Biointerfaces, 2009, 71(2), 200-207).

In one alternative for the present invention capture molecules are immobilized on the substrate in order to capture and immobilize the A-beta aggregates.

Preferably anti-A-beta antibodies are used as capture molecules.

In one alternative, the capture molecules are covalently bound to the substrate.

In a further alternative, the capture molecules are covalently bound to the coating, preferably dextran layer.

The anti-A-beta antibodies specifically bind one epitope of the A-beta aggregates. In one alternative of the present invention, the epitope has an amino acid sequence of the amino-terminal part of the A-beta peptide selected from the sub-segments A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8), for example of the human N-terminal epitope (with the following sequence: DAEFRHDSGYE (1-11, SEQ ID No. 3).

In one implementation of the present invention, the capture molecules (antibodies) are immobilized on the substrate, optionally after activation of the CMD-coated support by a mixture of EDC/NHS (200 and 50 mM respectively).

Remaining carboxylate terminal groups to which no capture molecules were bound can be deactivated.

For the deactivation of these carboxylate terminal groups on the CMD spacer, ethanolamine in DMSO is used. Before application of the samples, the substrates or supports are rinsed with PBS.

The sample to be assayed is incubated on the substrate thus prepared.

In one implementation of the present invention, the application of the sample is effected directly on the substrate (uncoated substrate), optionally by covalent bonding on the optionally activated surface of the substrate.

In one modification of the present invention, a pretreatment of the sample is effected by one or more of the following methods:
heating (at a temperature up to the boiling point of the sample)
one or more freeze-thaw cycles,
dilution with water or buffer,
treatment with enzymes, for example proteases, nuclease, lipases,
centrifugation,
precipitation,
competition with probes, in order to displace any anti-A-beta antibodies present.

In a further step, A-beta aggregates are marked by probes which are labeled for later detection.

In one modification of the present invention, anti-A-beta antibodies are used as probes. Capture molecules and probes can be identical.

In one implementation of the present invention, capture molecules and probes are different. Thus for example different anti-A-beta antibodies can be used as capture molecules and probes. In a further implementation of the present invention, capture molecules and probes are used which are identical to one another except for the possible dye marking. In one alternative of the present invention, various probes are used which are identical to one another except for the possible dye marking. In further alternatives of the present invention, at least 2 or more different capture molecules and/or probes are used, which are from different anti-A-beta antibodies and optionally also have different dye marking.

However, different molecules, such as for example different anti-A-beta antibodies, can also be used as capture molecules. Capture molecules can be specific amino acid sequences of the A-beta peptide, for example A-beta 1-40/42, pyroGlu 3-40/42 or pyroGlu 11-40/42.

Likewise, several, different molecules, such as for example different anti-A-beta antibodies, can be used as probes.

For subsequent quality control of the surface, for example uniformity of the coating with capture molecules, capture molecules labeled with fluorescent dyes can be used. For this, a dye which does not interfere with the detection is preferably used. Subsequent checking of the structure thereby becomes possible, and standardization of the assay results.

In one implementation of the present invention, anti-A-beta antibodies which bind specifically to the N-terminal epitopes of the A-beta peptide are used as probes.

For detection, the probes are labeled such that they emit an optically detectable signal, selected from the group consisting of fluorescence, bioluminescence and chemiluminescence emission and absorption.

In one alternative, the probes are labeled with dyes. Preferably, these are fluorescent dyes.

In one implementation of the present invention, at least 2, 3, 4, 5, 6 or more different probes are used. The probes can differ both with regard to their specific binding to the A-beta aggregates and also with regard to their different labeling, e.g. with fluorescent dyes.

Probes which are suitable to use FRET (Fluorescence Resonance Energy Transfer) as detection can also be combined with one another.

The use of several, different probes which are labeled with different fluorescent dyes increases the specificity of the correlation signal obtained in the measurement. In addition, masking of A-beta monomers also thereby becomes possible. The detection of A-beta monomers can in particular be excluded if probe and capture molecule are identical, or both recognize an overlapping epitope.

In one implementation of the present invention, probes which are specific for a defined A-beta aggregate species, such as for example A-beta (x-40,), A-beta (x-42) or pyroglutamate A-beta (3-x), pyroglutamate A-beta (11-x), are used. X is a whole natural number between 1 and 40 or 42, where those skilled in the art determine the length of the sequence to be used on the basis of their knowledge of the sequence of the A-beta peptide. In a further alternative, probes which are specific for defined A-beta aggregate forms, such as for example the commercially available antibodies “A-11” or “I-11”, can be used.

The exploitation or use of A-beta aggregate-specific or A-beta oligomer-specific probes is thus also a subject of the present invention. These specifically bind to a defined A-beta aggregate, or A-beta oligomer, preferably for the aforesaid species. Through the specific binding to a defined A-beta aggregate or A-beta oligomer the nature and/or size and the structure of the A-beta aggregate or A-beta oligomer can be determined.

A-beta aggregate-specific or A-beta oligomer-specific probes are thus also a subject of the present invention.

In a further alternative, A-beta peptides labeled with fluorescent dyes can be used as probes.

As samples to be tested, endogenous fluids or tissue can be used. In one implementation of the present invention, the sample is selected from spinal fluid (CSF), blood, plasma and urine. The samples can pass through different preparation steps known to those skilled in the art.

An advantage of the present invention is the possibility of determination of A-beta aggregates in untreated samples, preferably CSF.

A method for determining the composition, size and/or form of A-beta aggregates is thus also a subject of the present invention. In this, the process steps mentioned and described above are used.

The detection of the marked aggregates is effected by scanning or other types of surface imaging. The detection is preferably effected by confocal fluorescence microscopy or fluorescence correlation spectroscopy (FCS), in particular in combination with cross-correlation and single particle immunosolvent laser scanning assay and/or laser scanning microscope (LSM).

In one alternative of the present invention, the detection is effected with a confocal laser scanning microscope.

In one implementation of the present invention, a laser focus, such as for example is used in laser scanning microscopy, or an FCS (Fluorescence Correlation Spectroscopy System), is used for this, and the corresponding super resolution modifications such as for example STED or SIM. Alternatively to this, the detection can be effected with a TIRF microscope, and the corresponding super resolution modifications thereof, such as for example STORM or dSTORM.

Hence in the implementation of the invention methods which are based on a non-spatially resolved signal, such as ELISA or sandwich ELISA, are excluded.

In the detection, high spatial resolution is advantageous. In one implementation of the method according to the invention, so many data points are collected therein that the detection of one aggregate against a background signal which is for example caused by instrument-specific noise, other nonspecific signals or nonspecifically bound probes, is enabled. In this manner, as many values are read out (readout values) as spatially resolved events, such as for example pixels, are present. Through the spatial resolution, every event is determined against the respective background and thus represents an advantage compared to ELISA methods without spatially resolved signal.

In one alternative, several different probes are used in the method according to the invention. As a result, the information, i.e. the readout values, are multiplied, since for every point, for every aggregate or for every detection event, a separate information item is received, depending on the particular probe which yields the signal. Thus for each event the specificity of the signal is increased. Thus for every aggregate detected its composition, i.e. the nature of the aggregate, that is the composition of A-beta species, such as for example A-beta (1-40), A-beta (1-42), pyroglutamate A-beta (3-40/42, 11-40/42) or mixtures thereof, can also be determined.

The number of different probes is limited here only by the interference of the fluorescent dyes to be used. Thus 1, 2, 3, 4 or more different probe-dye combinations can be used.

Spatially resolved information is essential for the assessment according to the method described above. This can for example be the nature and/or intensity of the fluorescence. On assessment of these data for all probes used and detected, according to the invention the number of aggregates, and their form, size and/or their composition, are determined. Here information on the size of the oligomers can be obtained directly or indirectly, depending on whether the particles are smaller or larger than the spatial resolution of the imaging method used, in one implementation algorithms for background minimization can be used and/or intensity threshold values can be applied.

As the fluorescent dye, the dyes known to those skilled in the art can be used. Alternatively, GFP (Green Fluorescence Protein), conjugates and/or fusion proteins thereof, and quantum dots, can be used.

Through the use of internal or external standards, test results are objectively comparable with one another and therefore meaningful.

In one implementation of the present invention, an internal or external standard are used for the quantification of A-beta aggregates.

Based on the analysis of the distribution of the fluorescence intensity (FIDA-Fluorescence Intensity Distribution Analysis), the method according to the invention is a so-called surface FIDA.

By selection of the capture and probe molecules, it can be determined what size the oligomers must have in order to be able to contribute to the detection (signal).

In addition, with the method according to the invention the precise analysis of the small, freely diffusing A-beta aggregates is also possible. Because of their size, which lies below their resolution for optical microscopy, these small A-beta oligomers could with difficulty be distinguished from the background fluorescence (caused for example by unbound antibodies).

As well as the extremely high sensitivity, the method according to the invention also exhibits linearity over a large range with regard to the number of A-beta aggregates.

A further subject of the provisional invention is the use of the small, freely diffusing A-beta aggregates as biomarkers for the detection and the identification of protein aggregation diseases, in particular AD. The invention also relates to a method for the identification and/or detection of protein aggregation diseases, in particular AD, characterized in that a sample of a body fluid from a patient, preferably CSF, is analyzed with the above-described method according to the invention.

In one modification of the present invention, internal or external standards are used. Such standards for the quantification of oligomers or pathogenic aggregates which characterize a protein aggregation disease or an amyloid degeneration or protein misfolding disease, are characterized in that a polymer is constructed from polypeptide sequences which with regard to their sequence are identical with the endogenous proteins in the corresponding sub-segment or exhibit a homology with the endogenous proteins of at least 50% over the corresponding sub-segment, which characterize a protein aggregation disease or an amyloid degeneration or protein misfolding disease, wherein the polymers do not aggregate.

In the sense of the present invention, standard describes a generally valid and accepted, fixed reference quantity which is used for comparison and determination of properties and/or quantity, in particular for determining the size and quantity of pathogenic aggregates of endogenous proteins. The standard in the sense of the present invention can be used for the calibration of instruments and/or measurements.

In the sense of the present invention, amyloid degenerations and protein misfolding diseases can also be combined under the term “protein aggregation disease”.

Examples of such diseases and the endogenous proteins associated therewith are: A-beta and tau protein for AD, alpha synuclein for Parkinson's or prion protein for prion diseases, for example such as human Creutzfeld-Jakob disease (CJD), the sheep disease scrapie and bovine spongiform encephalopathy (BSE).

In the sense of the invention “homologous sequences” means that an amino acid sequence exhibits an identity with an amino acid sequence from an endogenous pathogenic aggregate or oligomers, which causes a protein aggregation disease, of at least 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. In the present description, instead of the term “identity”, the terms “homologous” or “homology” are used synonymously. The identity between two nucleic acid sequences or polypeptide sequences is calculated by comparison by means of the program BESTFIT based on the algorithm of Smith, T. F. and Waterman, M. S (Adv. Appl. Math. 2: 482-489 (1981)) with setting of the following parameters for amino acids: gap creation penalty: 8 and gap extension penalty: 2; and the following parameters for nucleic acids: gap creation penalty: 50 and gap extension penalty: 3. Preferably the identity between two nucleic acid sequences or polypeptide sequences is defined by the identity of the nucleic acid sequence/polypeptide sequence over the whole particular sequence length, as calculated by comparison by means of the program GAP based on the algorithm of Needleman, S. B. and Wunsch, C. D. (J. Mol. Biol. 48: 443-453) with setting of the following parameters for amino acids: gap creation penalty: 8 and gap extension penalty: 2; and the following parameters for nucleic acids gap creation penalty: 50 and gap extension penalty: 3.

Two amino acid sequences are identical in the sense of the present invention if they possess the same amino acid sequence.

The term “corresponding sub-segment” of endogenous proteins should be understood to mean that peptide sequence which according to the definitions according to the invention exhibits an identical or with the stated percentage homologous peptide sequence of a monomer, from which the standards according to the invention are constructed.

It is essential for the standards according to the invention that the standards do not aggregate, preferably due to the use of monomeric sequences which do not aggregate, since the “corresponding sub-segment” of endogenous proteins is not responsible for the aggregation, or the groups responsible for the aggregation do not aggregate because of blocking.

Aggregates in the sense of the present invention are
particles which consist of several, preferably identical building blocks which are not bound covalently to one another and/or
non-covalent agglomerations of several monomers.

In one implementation of the present invention, the standards have a precisely defined number of epitopes which are covalently linked to one another (directly or via amino acids, spacers and/or functional groups) for the binding of the relevant probes. Probes in the sense of the invention are selected from the group consisting of: antibodies, nanobody and affibody. Furthermore, probes are all molecules which possess adequate binding specificity for the aggregate to be detected, e.g. dyes (thioflavin T, Congo red, etc.).

The number of epitopes is determined by using a polypeptide sequence which with regard to its sequence is identical with that sub-segment of the endogenous proteins which forms an epitope or exhibits homology of at least 50% with this sub-segment, and also possesses the biological activity of the epitope. A polypeptide sequence thus selected is incorporated in the desired number during the construction of the standard according to the invention and/or linked together according to the invention.

The standards according to the invention are polymers which are made up of the polypeptide sequences, preferably epitopes, described above, optionally containing further components.

In a further implementation of the present invention, the above-described polypeptide sequences, preferably epitopes, and/or homologs thereof with the biological activity of the corresponding epitope, represent the equal or greatest number of monomers based on the number in each case of one of the residual monomer species of the standard and/or based on the number of all other monomers.

In a further implementation of the present invention, the epitopes are epitopes of the A-beta peptide selected from the sub-segments A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8), for example of the human N-terminal epitope (with the following sequence: DAEFRHDSGYE (1-11; corresponds to SEQ ID No. 3).

PyroGlu is the abbreviation for a pyroglutamate which is located at position 3 and/or 11 of the A-beta peptide, and is preferably based on a cyclization of the N-terminal glutamate.

The standard molecule according to the invention is a polymer of the polypeptide sequences defined above. Oligomer in the sense of the invention is a polymer formed from 2, 3, 4, 5, 6, 7, 8, 9, 10, 11, 12, 13, 14, 15, 16, 17, 18, 19 or 20 monomers (monomer should be understood to mean the aforesaid polypeptide sequence), or multiples thereof, preferably 2-16, 4-16, 8-16, particularly preferably 8 or 16, or multiples thereof.

The standards according to the invention are thus oligomers or polymers according to the invention.

In one alternative of the present invention, the standards are water-soluble.

In one alternative of the present invention, the standards according to the invention are made up of identical polypeptide sequences.

In one alternative of the present invention, the standards according to the invention are made up of different polypeptide sequences.

In one alternative of the present invention, such above-defined polypeptide sequences are concatenated in a linear conformation.

In one alternative of the present invention, such above-defined polypeptide sequences are concatenated in a branched oligomer according to the invention.

In one alternative of the present invention, such above-defined polypeptide sequences are concatenated in a cross-linked oligomer according to the invention. Branched or cross-linked oligomers according to the invention can be produced by linking individual building blocks by means of lysine or by means of click chemistry. As described above, the standards according to the invention, that is the oligomers or polymers according to the invention, in addition to the polypeptide sequences, preferably epitopes, present in precisely defined number, can further contain additional amino acids, spacers and/or functional groups, via which the polypeptide sequences, preferably epitopes, are covalently linked to one another.

In one alternative, the direct linkage of the polypeptide sequences, preferably epitopes with cysteine, in particular by disulfide bridging by cysteines is excluded (in order to avoid reducing agents removing the bridging). Likewise in a further modification, direct linkage of the spacers with the polypeptide sequence on the one hand and with cysteine on the other is excluded.

In one alternative, the invention relates to a standard molecule, containing or made up of copies of the amino-terminal part of the A-beta peptide, selected from the sub-segments A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8), for example of the human N-terminal epitope (with the following sequence: DAEFRHDSGYE (1-11).

The duplication of the epitopes via functional groups can be performed before or after the synthesis of the individual building blocks. The covalent linkage of the polypeptide sequences is characteristic for the standards according to the invention.

The polypeptide sequences to be used according to the invention can be identical with the sequence of the A-beta full-length peptide or exhibit a homology of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% with the sequence of the A-beta full-length peptide.

Alternatively, polypeptide sequences which are identical with a sub-segment of the A-beta full-length peptide, or exhibit a homology of 50, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% with a sub-segment of the A-beta full-length peptide, are also used for constructing the standard molecules according to the invention.

Essential for the sequences used according to the invention is their property of not aggregating (or only in a controlled manner depending on the conditions) and/or their the activity as epitope.

In a further implementation of the present invention, the standards are constructed as dendrimers. The dendrimers according to the invention are constructed of the above-described polypeptide sequences to be used according to the invention and can contain a central scaffold molecule. Preferably the scaffold molecule is a streptavidin monomer, particularly preferably a polymer, in particular tetramer.

In one modification, the dendrimers according to the invention contain polypeptide sequences which possess a sequence which is identical with a sub-segment of the A-beta peptide, or exhibits at least 50% homology to the corresponding sub-segment.

According to the invention, the term at least 50% homology should also be understood to mean a higher homology selected from the group consisting of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In one implementation of the invention, standards, advantageously with higher solubility in the aqueous than pathogenic aggregates or oligomers of endogenous proteins, are formed of polypeptide sequences which are identical with the N-terminal region of the A-beta peptide or exhibit at least 50% homology thereto. According to the invention, the N-terminal region of an A-beta polypeptide should be understood to mean the amino acid sequence A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8).

A standard molecule according to the invention can contain epitopes for at least 2, 3, 4, 5, 6, 7, 8, 9, 10 or more different probes.

Epitopes characteristic for different probes can be incorporated into the standards according to the invention by using polypeptide sequences which are identical with different regions of the A-beta peptide or exhibit 50% homology thereto, but possess the activity of the corresponding epitope.

In one implementation, polypeptide sequences which are identical or exhibit 50% homology with the N-terminal region of the A-beta polypeptide and polypeptide sequences which are identical or exhibit at least 50% homology with the C-terminus of the A-beta polypeptide are used for this.

In one implementation of the present invention, the standard molecules contain so-called spacers.

A spacer should be understood to mean a molecule which is incorporated into the standard molecule via covalent bonds, and possesses defined physical and/or chemical properties, through which the properties of the standard molecules are modified. In one implementation of the standards according to the invention, hydrophilic or hydrophobic, preferably hydrophilic spacers are used. Hydrophilic spacers are selected from the group of molecules made up of polyethylene glycol, sugars, glycerin, poly-L-lysine or beta-alanine.

In one alternative of the present invention, the standards according to the invention contain (further) functional groups.

Functional groups should be understood to mean molecules which are covalently bound to the standard molecules. In one modification, the functional groups contain biotin groups. As a result, strong covalent bonding to streptavidin is enabled.

Standard molecules containing biotin groups can thus be bound to molecules containing streptavidin groups. If the standard molecules according to the invention contain biotin and/or streptavidin groups, larger standards can thus be assembled or several optionally different standard molecules, bound onto one scaffold.

In a further alternative of the present invention, the standard molecules contain dyes for spectrophotometric determination and/or aromatic amino acids. Aromatic amino acids are e.g. tryptophan, tyrosine, phenylalanine or histidine, or selected from this group. Through the incorporation of tryptophan, spectrophotometric determination of the concentration of standards in solution is enabled.

A further subject of the present invention are dendrimers containing polypeptides which with regard to their sequence are identical in the corresponding sub-segment with the endogenous proteins or exhibit a homology of at least 50% over the corresponding sub-segment with the endogenous proteins which characterize a protein aggregation disease.

The dendrimers according to the invention can contain any of the above-described features of the standards or any desired combination thereof.

In one alternative of the present invention, these are:
dendrimers containing a precisely defined number of epitopes for the covalent binding of probes,
dendrimer containing epitopes of the A-beta peptide,
dendrimer characterized in that it possesses a higher solubility in the aqueous than the pathogenic aggregates of endogenous proteins which characterize a protein aggregation disease,
dendrimer containing functional groups,
dendrimer containing at least one spacer molecule and/or dendrimer containing dyes for spectrophotometric determination and/or aromatic amino acids.

According to the invention, the dendrimers have radial symmetry.

In one modification, the branching of the first generation of the dendrimer is effected via lysine, in particular three lysine amino acids.

In a further alternative of the present invention, in the standards, in particular dendrimers, the polypeptide sequences, preferably epitopes, are linked, in particular covalently bound to one another or to other components of the standard such as amino acids, spacers and/or functional groups and/or other above-described components not via a bond to a sulfur atom, not via a thioether bond and/or not via cysteine (optionally by disulfide bridging via cysteine). Likewise in a further modification, the polypeptide sequences, preferably epitopes, and a spacer bound thereto are linked, in particular covalently bound to one another or to other components of the standard such as amino acids, spacers and/or functional groups and/or other described components not via a bond to a sulfur atom, not via a thioether bond and/or not via cysteine.

The present invention further relates to a method for the production of a standard, as described above.

In one implementation, the standard according to the invention is produced by peptide synthesis or recombinant methods which are known to those skilled in the art.

A further subject of the present invention is the use of an above-described standard or an above-described dendrimer for quantifying pathogenic aggregates or oligomers of endogenous proteins which characterize a protein aggregation disease.

In one implementation of the invention, the standard is used to quantify A-beta oligomers.

According to the invention, the oligomers or polymers according to the invention are used as a standard in a method for quantifying pathogenic aggregates or oligomers of endogenous proteins which characterize a protein aggregation disease or an amyloid degeneration or protein misfolding disease.

The standards according to the invention are used in one implementation of the present invention for calibration in the surface FIDA method, Elisa (sandwich Elisa) or FACS.

In another embodiment, the present invention relates to a kit which comprises standard according to the invention. The compounds and/or components of the kit of the present invention can be packed in containers optionally with/in buffers and/or solution. Alternatively, a number of components can be packed in the same container. In addition to this or alternatively to this, one or more of the components could be adsorbed on a solid support, such as for example a glass plate, a chip or a nylon membrane or on the well of a microtiter plate. Further, the kit can contain directions for the use of the kit for any one of the embodiments.

In one alternative of the present invention, the standards for quantifying pathogenic aggregates or oligomers of endogenous proteins are used in that:
in a first step the standards or the dendrimers are marked with probes and the number of the probe bound to the standards or dendrimers is determined,
in a second step pathogenic aggregates or oligomers of endogenous proteins which characterize a protein aggregation disease are marked with probes, the number of the probes binding in each case to a pathogenic aggregate or oligomer is determined,
in a third step the number of probes binding respectively to a standard or dendrimer from step 1 is compared with that from step 2, and
in a fourth step the number and the size of the oligomers from the body fluid is thereby determined.

In one modification of the present invention, the standards according to the invention, preferably dendrimers, are used for the calibration of the surface FIDA method. In a first step, endogenous pathogenic aggregates from body fluids, e.g. A-beta aggregates, are immobilized on a glass surface by a probe. In the case of A-beta aggregates an N-terminal capture probe can be used for this. After the immobilization, the aggregates are marked by two different probes. In the case of A-beta aggregates, A-beta antibodies which are both bound via an N-terminal binding epitope are for example used. The detection probes are marked with preferably different fluorescent dyes. They thereby become visible under the microscope, e.g. laser scanning microscope.

According to the invention, monomer detection of endogenous polypeptides is excluded since in the test system three different or three differently marked probes which bind to a similar or identical epitope are used. Alternatively or in addition, the detection of monomers can be excluded in that signals with a lower intensity are not assessed because of an intensity cut-off. Since larger aggregates possess several binding sites for the two probes with different marked dyes, monomer detection can alternatively or additionally be excluded by cross-correlation of these signals.

The standards according to the invention can be used as internal or external standards in the assay.

Also a subject of the present invention is a kit for the selective quantification of A-beta aggregates according to the above-described method. Such a kit can contain one or more of the following components:
glass substrate which is coated with a hydrophobic substance, preferably dextran, preferably carboxymethyl dextran;
standard;
capture molecule;
probe;
substrate with capture molecule.

The compounds and/or components of the kit of the present invention can be packed in containers optionally with/in buffers and/or solution. Alternatively a number of components can be packed in the same container. In addition to this or alternatively to this, one or more of the components could be absorbed on a solid support, such as for example a glass plate, a chip or a nylon membrane or on the well of a microtiter plate. Further, the kit can contain directions for the use of the kit for any one of the embodiments.

In a further modification of the kit, the above-described capture molecules are immobilized on the substrate. In addition, the KIT can contain solutions and/or buffer. To protect the dextran surface and/or the capture molecules immobilized thereon, these can be covered with a solution or a buffer.

A further subject of the present invention is the use of the method according to the invention for the diagnosis, early diagnosis and/or prognosis of AD.

A further subject of the present invention is the use of the method according to the invention for monitoring therapies of AD and for monitoring and/or checking the effectiveness of active substances and/or therapies. This can be used in clinical tests, studies and also in therapy monitoring. For this, samples are assayed according to the method according to the invention and the results compared.

A further subject of the present invention is the use of the method according to the invention and the biomarkers for deciding whether a person is accepted in a clinical study. For this, samples are assayed according to the method according to the invention and the decision taken with reference to a limit value.

A further subject of the present invention is a method for determining the effectiveness of active substances and/or therapies by means of the method according to the invention, in which the results from samples are compared with one another. The samples are body fluids withdrawn before or after, or at different times after administration of the active substances or implementation of the therapy. On the basis of the results, active substances and/or therapies are selected, through which a reduction in the A-beta aggregates occurred. According to the invention the results are compared with a control which was not subjected to the active substance and/or therapy.

### EXAMPLES

### I. Determination of A-beta Oligomers (A-Beta Aggregates) in CSF

### 1. Substrate Preparation

Glass supports were cleaned in an ultrasonic bath for 15 minutes. The surface was rinsed three times with water and dried in a current of nitrogen gas. The cleaned supports were immersed in a 3:1 (V/V) mixture of concentrated sulfuric acid and hydrogen peroxide for at least 30 minutes in order to activate the hydroxyl groups. It was then rinsed with water until the rinse water had a neutral pH. In a second rinsing step 99% ethanol was used and then the support dried in the current of nitrogen gas. The glass supports were immersed in a solution of 1-7% 3-amino-propyltriethoxysilane (APTES) in dry toluene for 1 to 4 hours. Good results were achieved with 5% APTES solution and an incubation time of 2 hours. Then the slides were rinsed with acetone and water and dried in a current of nitrogen gas.

For coating with dextran, the glass surface was hydroxylated and then activated with amino groups. Carboxymethyl dextran (CMD) was dissolved in water at a concentration of 10 mg per ml and mixed with N-ethyl-N-(3-dimethylaminopropyl) carbodiimide (EDC) (200 mM) and N-hydroxysuccinimide (NHS), (50 mM). After a preincubation of 10 minutes, the solution was incubated for a further 2 hours at room temperature. Then the glass supports were washed with water.

### 2. Immobilization of Antibodies as Capture Molecules on the Coated Substrate.

A second activation of the surface was effected with a solution of EDC/NHS (200 or 50 mM) for 5 minutes. The solution of the antibody was added to this and incubated for 2 hours at 4° C. As a result the antibodies were covalently bound to the CMD-coated glass surface. In order then to deactivate remaining active carboxyl terminal groups on the CMD spacer, this was incubated with 1M ethanolamine in DMSO for 15 minutes. The substrate was then washed three times with PBS.

### 3. Immobilization of A-Beta Aggregates on the Pretreated Substrate

The sample to be assayed was incubated for 1 hour on the substrate, and this was then washed twice with TBST (0.1%) (WAN), Tween-20 in TBS buffer, TBS: 50 nM Tris-HCl, 0.15 M nacl, pH 7.4).

4. Linking of the Samples with Fluorescent Dye for their Labeling

Nab 228, antimouse Alexa 633 and 6 E10 Alexa 488 antibodies were used. The Nab 228 antibodies were labeled with a KIT (Fluorescence labeling KIT Alexa-647, Molecular Probes, Karlsruhe, Germany) according to the manufacturer's instructions. The labeled antibodies were stored in PBS containing 2 mM sodium azide at 4° degrees in the dark.

5. Marking of the Aggregates with the Probes

The quantity of the antibody used was dependent on the desired degree of marking. The probes were added and incubated for 1 hour at room temperature, then washed five times with TBST and twice with TBS.

### 6. Detection of the Aggregates and Assay of the Samples

The measurement was effected with a confocal laser scanning microscope LSM 710 (Carl Zeiss, Jena, Germany). The microscope was equipped with an argon ion laser and three helium-neon lasers. The laser beams were focused on a diffraction-limited spot of a volume of 0.25 femtoliters. The fluorescence intensity of an area of 1000×1000 pixels was determined. Since different probes were used, a colocalization analysis was performed. In order to obtain representative values, this area was measured at several sites on the support.

The measurement was made with ZEN 2008 software from Carl Zeiss, Jena, Germany.

### 7. Analysis of CSF Samples

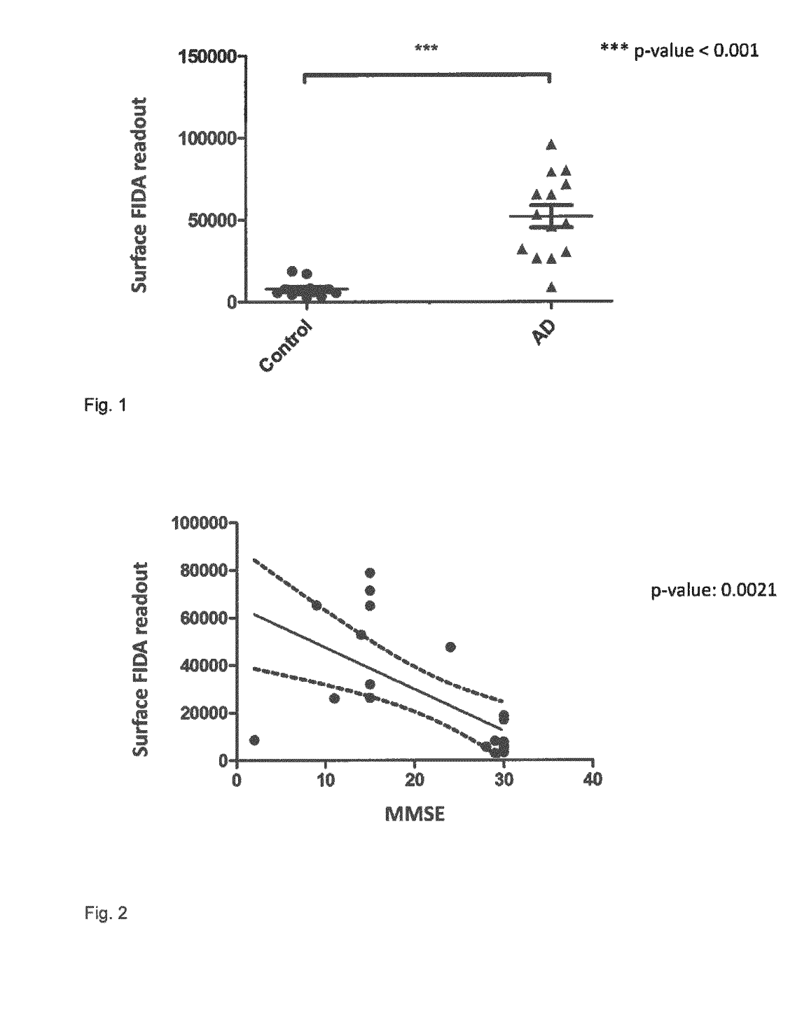

26 samples of CSF from different patients were analyzed with the method according to the invention. The samples derive respectively from 14 AD patients and 12 control patients (healthy with regard to protein aggregation diseases, of different age). The results are summarized in FIG. 1. The results show that a marked distinction between the groups is possible. The average of A-beta oligomers in the AD group was significantly higher than in the control group.

8. Correlation with MMSE

The results of the analysis according to the invention were compared with an MMSE (Mini Mental Status Test) of the donors. These results are summarized in FIG. 2. From this, the correlation between the assessment of the MMSE test and the evaluation of the analysis according to the invention becomes clear.

### II. Detection of Aggregate Standards

### 1. Preparation of Aggregate Standards

In a practical example, an A-beta oligomer standard was constructed which exhibited 16 epitopes for N-terminal-binding A-beta antibodies (epitope corresponds to A-beta-(1-11), sequence: DAEFRHDSGYE).

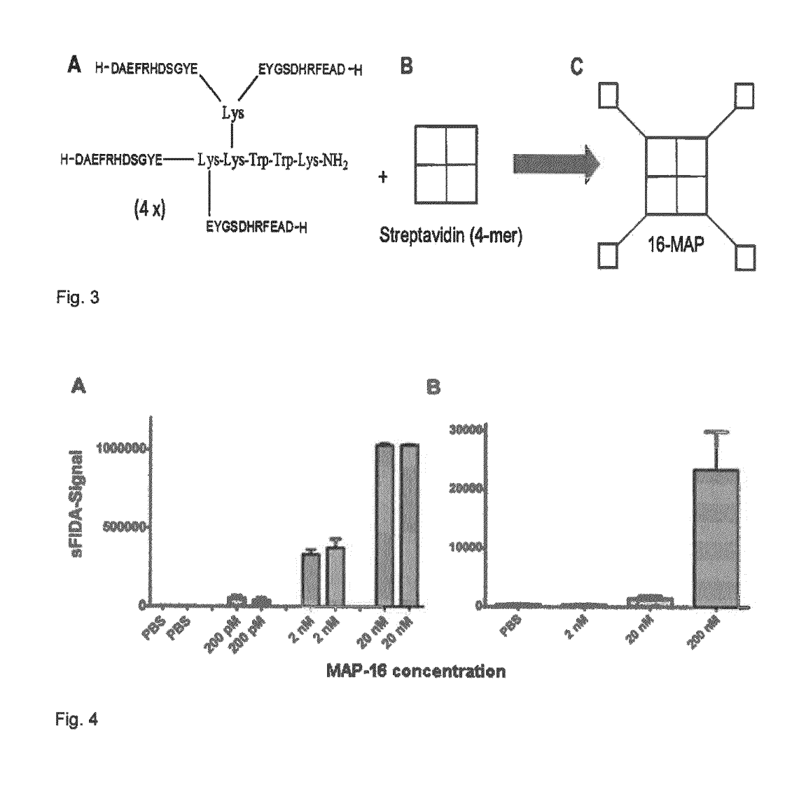

Firstly, a multiple antigen peptide (MAP) was synthesized which consisted of four N-terminal A-beta epitopes A-beta1-11. These were coupled in accordance with FIG. 3A to a triple lysine core, which for the precise determination of the MAP concentration by UV/VIS spectroscopy contained two tryptophans. In addition, a biotin tag was attached N-terminally. This was used for the coupling of respectively four 4-MAP units to a streptavidin tetramer, shown under B in FIG. 3. After incubation of 4-MAP and streptavidin 16-MAP was formed, as shown under C in FIG. 3. 16-MAP was separated from other components of the incubation mixture by size exclusion chromatography.

Next, MAP-16 was serially diluted in PBS and used in the sFIDA test for the detection of A-beta oligomers.

### 2. Glass Plate Preparation

Glass microtiter plates were cleaned in an ultrasonic bath for 15 minutes and then treated with a plasma cleaner for 10 mins. For the activation of the glass surface, the wells were incubated in 5M NaOH for at least 3 hours, rinsed with water and then dried in the current of nitrogen gas. For the coating with dextran, the glass surface was hydroxylated and then activated with amino groups. For this, the glass plates was incubated overnight in a solution of 5M ethanolamine in DMSO. Next, the glass plates were rinsed with water and dried in a current of nitrogen gas. Carboxymethyl dextran (CMD) was dissolved in water at a concentration of 20 mg per ml and mixed with N-ethyl-N-(3-dimethylaminopropyl) carbodiimide (EDC), (200 mM) and N-hydroxysuccinimide (NHS), (50 mM). After a preincubation of 10 minutes, the solution was incubated for a further 2 hours at room temperature. Then the glass plates were washed with water.

### 3. Immobilization of Antibodies as Capture Molecules on the Coated Glass

A second activation was effected with a solution of EDC/NHS (200 or 50 mM) for 5 minutes. The solution of the antibodies was added to this and incubated for 2 hours at 4° C. As a result, the antibodies were covalently bound onto the CMD-activated glass surface. In order then to deactivate remaining active carboxyl terminal groups on the CMD spacer, this was incubated for 5 minutes with 1M ethanolamine in DMSO. The glass was then washed three times with PBS.

### 4. Immobilization of MAP-16 on the Pretreated Glass

The MAP-16-containing sample to be assayed was incubated for 1 hour on the glass, then washed three times with TBST (0.1%) (WAN), Tween-20 in TBS buffer, TBS: 50 nM Tris-HCl, 0.15 M NaCl, pH 7.4).

5. Labeling of the Probes with Fluorescent Dye

6E10 Alexa-488 antibodies and IC-16 antibodies were used. The IC16 antibodies were marked with a kit (Fluorescence labeling KIT Alexa-647, Molecular Probes, Karlsruhe, Germany) according to the manufacturer's instructions. The labeled antibodies were stored in PBS containing 2 mM sodium azide at 4° C. in the dark.

6. Marking of the Aggregates with the Probes

The probes were added and incubated for 1 hour at room temperature, then washed five times with TBST and twice with water.

### 7. Detection of the Aggregate Standard

The measurement was effected with a confocal laser scanning microscope LSM 710 (Carl Zeiss, Jena, Germany). The microscope was equipped with an argon ion laser and three helium-neon lasers. The measurements were effected in tile scan model, in which adjacent surfaces in a well are measured and assembled to an image. Each tile scan contained 3×2 individual images, and each image had an area of 213×213 μm.

Alternatively, the measurements were effected on a TIRF microscope (TIRF=total internal reflection) consisting of an inverted microscope DMI 6000, a laser box and a Hamamatsu EM-CCD C9100 camera. In the tile scan mode 3×3 individual images each with a size of 109.9×109.9 μm were.

The assessment was effected with the software “Image J” ((http://rsbweb.nih.gov/ij/). Through the use of different probes, a colocalization analysis could be performed. For this, firstly a cut-off value, defined by a negative control without MAP-16, was subtracted from the intensity values of the individual pixels. Next, the number of colocalized pixels whose intensity was greater than zero was added.

FIG. 4 shows the results of the measurements. It can clearly be discerned that the sFIDA signal, i.e. the quantity of the colocalized pixels, correlates with the concentration of the MAP-16 molecules.

### III. Comparison of A-Beta Aggregates (A-Beta Oligomers) with A-Beta Monomers

1. Determination by sFIDA

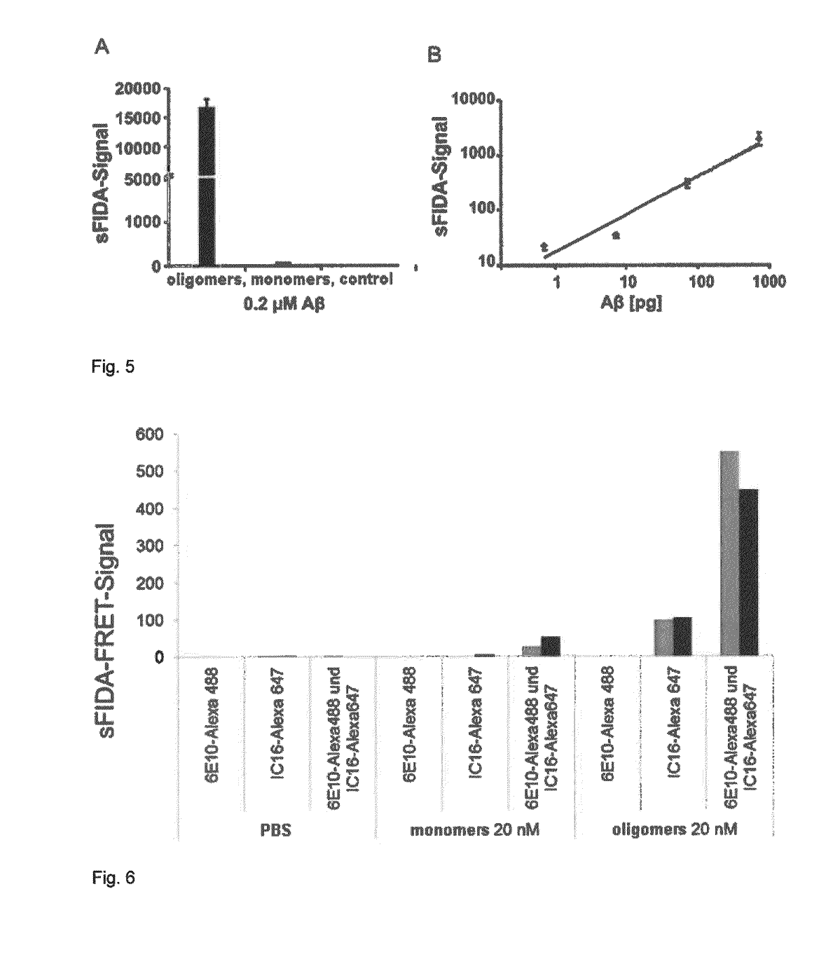

In order to be able to exclude the possibility that A-beta monomers are also detected by sFIDA and thus the signal of the A-beta oligomers is distorted, A-beta monomers and oligomers, consisting of synthetic A-beta, were prepared according to a protocol of Johannson et al., FEBS J. 2006, 273, pages 2618-2630, and tested with the system. In addition, the A-beta oligomers were serially diluted in PBS and the linearity of the test was checked in a concentration series. The measurements were performed as already described above, a Zeiss LSM 710 microscope was used for the detection and 2×25 images each with a size of 213×213 μm and 1024×1024 pixels were recorded. The results are shown in FIG. 5. A-beta oligomers resulted in a clear sFIDA signal, however this was not the case with A-beta monomers. On the basis of FIG. 5B it can be discerned that the sFIDA signal correlated with the concentration of the A-beta oligomers and moreover a very low A-beta oligomer concentration was necessary to result in a positive signal.

### 2. FRET Measurement

In order to establish whether for sFIDA another signal than the previously selected number of cross-correlated pixels can also be generated, FRET measurements were performed. FRET stands for Förster resonance energy transfer. In FRET the energy of an excited fluorochrome is transferred to a second fluorochrome. The FRET intensity depends inter alia on the distance between donor and acceptor and can be observed in the range of up to 10 nm. Thus it should be possible to use FRET in sFIDA in order to distinguish A-beta monomers from A-beta oligomers. Binding an anti-A-beta antibody (e.g. 6E10-Alexa488) coupled with a donor dye and an anti-A-beta antibody (e.g. IC-16-Alexa647) coupled with an acceptable dye suitable for this in direct proximity to one another onto an A-beta oligomer, FRET becomes possible due to the spatial proximity. It should statistically be rather improbable that 6E10-Alexa-488 and IC-16-Alexa647 bind to two A-beta monomers which by chance were immobilized at a distance of less than 10 nm from one another. This probability can be reduced to zero if antibodies which possess an epitope overlapping with the capture antibody are used for the detection. For the experiment, A-beta monomers and A-beta oligomers were prepared by size exclusion chromatography and immobilized and for the sFIDA measurements, as described above. In the subsequent measurements on a Leica fluorescence microscope, the fluorochromes were excited with a wavelength of 488 nm and the FRET emission detected at a wavelength of 705 nm. As controls, two samples were also measured in each of which only one fluorescent dye-coupled antibody was added.

As is clear in FIG. 6, the measurements resulted in a FRET signal only with A-beta oligomers, but not with A-beta monomers or controls.

### IV. Determination of A-Beta Aggregates in the Spinal Fluid of Alzheimer's Mouse Models

In further investigations, it was investigated whether sFIDA is also suitable for detecting A-beta aggregates in the spinal fluid of Alzheimer's mouse models and if so, at what dilution. For the experimental procedure, the spinal fluid from APP/Ps1 mice and non-transgenic control animals was diluted 1:10, 1:50 and 1:250 in PBS buffer and assayed by means of sFIDA.

The experimental procedure corresponds to that described above, however the measurements were performed on a Leica LSM. It was found that in one of the two samples from transgenic mice even at 250-fold dilution a markedly higher sFIDA signal could still be detected than with the samples from non-transgenic control animals. Per well, 25 areas (each 246 μm) with 1024×4024 pixels, i.e. 16% of the well area, were assayed.

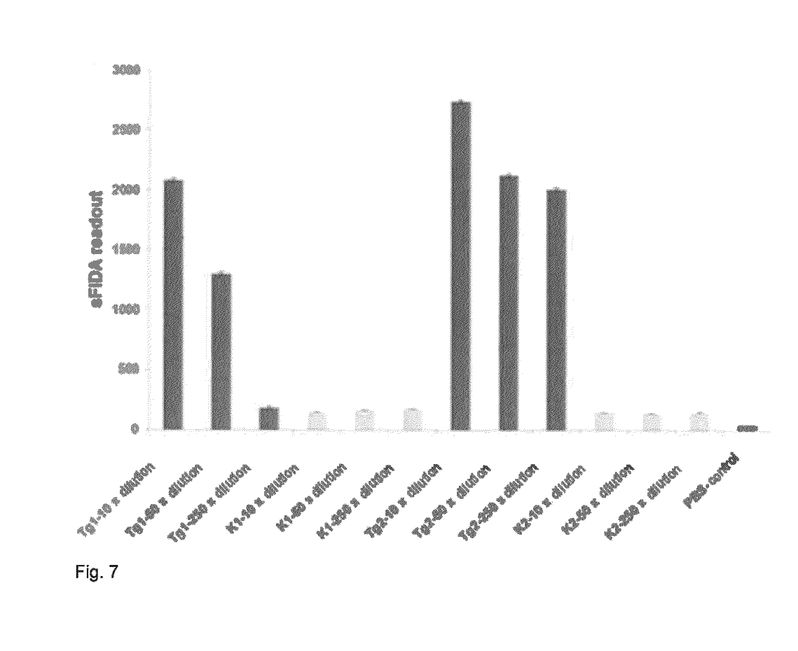

The results are shown in FIG. 7. They show that sFIDA is not only suitable for early diagnosis in man, but is also suitable for monitoring the effectiveness of a therapy in practical studies.

### DESCRIPTION OF DIAGRAMS

FIG. 1

Determination of A-beta aggregates in CSF from patients

FIG. 2

Correlation of the results from FIG. 1 with MMSE

FIG. 3:

Construction of an Aβ oligomer standard with 16 epitopes for N-terminal-binding Aβ antibodies which correspond to the first 11 amino acids of Aβ (sequence: DAEFRHDSGYE). A) 4-MAP was synthesized, consisting of 4 N-terminal Aβ epitopes 1-11 coupled to a threefold lysine core which contained two tryptophans for the concentration determination by UVNIS spectroscopy. B and C) For the production of 16-MAP in each case four 4-MAP were coupled via a streptavidin teramer. MAP-16 was separated from other components of the incubation mixture by means of size exclusion chromatography.

FIG. 4:

sFIDA measurements of MAP-16 at various concentrations, diluted in PBS buffer. PBS buffer with no MAP-16 was used as the negative control. A) The measurements were performed on a laser scanning microscope (Zeiss LSM 710). B). The measurements were performed on a TIRF microscope (Leica).

FIG. 5:

A) sFIDA is non-sensitive towards Aβ monomers, but B) detects Aβ oligomers concentration-dependently, linearly and with high sensitivity. Aβ monomers and oligomers were prepared from synthetic Aβ by means of size exclusion chromatography and diluted in PBS buffer.

FIG. 6:

sFIDA measurements with FRET signal on Aβ monomers and Aβ oligomers. PBS was used as the negative control. As further controls, samples were assayed, in each of which only one dye-coupled antibody was added. The donor dye was Alexa 488 coupled to Aβ antibody 6E10, and the acceptor dye was Alexa 647 coupled to Aβ antibody IC-16.

FIG. 7:

sFIDA detection of Aβ oligomers in the spinal fluid of transgenic (Tg) Alzheimer's mouse models (APP/PS1) and non-transgenic control animals (K). As the negative control, a pure buffer sample was used.

1.-27. (canceled)

28. A method for selectively quantifying and/or characterizing A-beta aggregates in a sample, which method comprises:
(a) applying a sample to be tested onto a substrate,
(b) adding probes labeled for detection, which probes label A-beta aggregates by specifically binding to the aggregates, and
(c) detecting labeled A-beta aggregates,
it being possible for (b) to be performed prior to (a).

29. The method of claim 28, wherein prior to (a) scavenger molecules are immobilized on the substrate.

30. The method of claim 28, wherein the sample is pretreated.

31. The method of claim 28, wherein glass substrate is employed.

32. The method of claim 28, wherein the substrate comprises a hydrophilic coating.

33. The method of claim 28, wherein the substrate is coated with dextran.

34. The method of claim 28, wherein scavenger molecules are covalently bound to the substrate or to a coating thereof.

35. The method of claim 34, wherein the scavenger molecules are labeled with a fluorescent dye.

36. The method of claim 34, wherein the scavenger molecules are anti-A-beta antibodies.

37. The method of claim 36, wherein the anti-A-beta antibodies specifically bind an epitope of the A-beta aggregate.

38. The method of claim 28, wherein A-beta peptide-specific probes are used.

39. The method of claim 28, wherein the probes are fluorescent dye-labeled anti-A-beta antibodies.

40. The method of claim 28, wherein two or more different probes are used.

41. The method of claim 28, wherein two or more probes with differently labeled fluorescent dyes are used.

42. The method of claim 28, wherein at least one probe is an anti-A-beta antibody which specifically binds to an N-terminal epitope of the A-beta peptide.

43. The method of claim 28, wherein (c) comprises spatial resolution fluorescence microscopy.

44. The method of claim 28, wherein (c) comprises one or more of confocal fluorescence microscopy, fluorescence correlation spectroscopy (FCS), optionally in combination with cross-correlation and single particle immunosolvent laser scanning assay, laser scanning microscopy (LSM), Wetfeld microscopy, TIRF microscopy, and the corresponding super resolution modifications STED, SIM, STORM and dSTORM.

45. The method of claim 44, wherein in (c) sufficient data points for enabling detection of a single aggregate against a background signal are collected.

46. The method of claim 45, wherein a number of read-out values corresponds to a number of spatially resolved events present.

47. The method of claim 28, wherein the sample comprises one or more of spinal fluid (CSF, cerebrospinal fluid), blood, urine.

48. The method of claim 28, wherein an internal or external standard is used for quantifying A-beta aggregates.

49. The method of claim 48, wherein a standard for quantifying A-beta aggregates comprises non-aggregating polymers constructed from polypeptide sequences which with respect to their sequence are identical in a sub-segment or exhibit homology of at least 50% across a sub-segment with endogenous proteins that cause a protein aggregation disease or an amyloid degeneration or protein misfolding disease.

50. A kit for selectively quantifying A-beta aggregates, wherein the kit comprises one or more of:
a glass substrate coated with a hydrophobic substance;
a standard;
scavenger molecules;
a probe;
a substrate comprising scavenger molecules;
solutions; and
a buffer.

51. A method for determining the effectiveness of an active substance and/or therapy for treating AD, wherein the method comprises performing the method of claim 28, comparing active substances and/or therapies with one another in terms of their effect on A-beta aggregate formation, and selecting active substances and/or therapies which exhibit lower A-beta aggregate formation compared with a control.

52. A method for deciding on the acceptance of an individual into a clinical study or test, wherein the method comprises quantifying and/or characterizing A-beta aggregates according to the method of claim 28 and comparing a measured value with a threshold value.

53. A probe, wherein the probe is at least one of A-beta aggregate-specific and A-beta oligomer-specific.

54. A method of using the probe of claim 53, wherein the method comprises specifically binding the probe to a defined A-beta aggregate or A-beta oligomer.
